(19) United States
(12) Patent Application Publication
(10) Pub. No.: US 2025/0345170 A1
(43) Pub. Date: Nov. 13, 2025

(54) METHODS OF ISOLATING AND USING DUA'S LAYER

(71) Applicant: Lions World Vision Institute, Inc., Tampa, FL (US)

(72) Inventors: Eric Abdullayev, Tarpon Springs, FL (US); Arthur C. Kurz, Safety Harbor, FL (US)

(21) Appl. No.: 19/200,876

(22) Filed: May 7, 2025

Related U.S. Application Data
(60) Provisional application No. 63/643,513, filed on May 7, 2024.

(51) Int. Cl. A61F 2/14; A61B 17/00; A61B 17/32; A61F 9/008; A61L 27/36

(52) U.S. Cl. CPC A61F 2/1451; A61F 9/00831; A61F 9/0084; A61L 27/3604; A61L 27/3666; A61B 2017/00539; A61B 2017/00544; A61B 2017/320044; A61F 2009/00872; A61F 2240/001; A61L 2430/16

(57) ABSTRACT

One method of isolating the Dua's layer includes separating the Descemet's membrane from the cornea using a hydrodissection technique, removing the Descemet's membrane from the cornea, and separating the Dua's layer from the cornea. A second method of isolating the Dua's layer includes simultaneously separating the Dua's layer from the stroma and separating the Dua's layer from the Descemet's membrane. A third method of isolating the Dua's layer includes dissecting the Dua's layer from the cornea using femtosecond laser. A fourth method of isolating the Dua's layer manually dissecting the Dua's layer from the cornea using forceps. The isolated Dua's layer can be preserved as either a fresh graft for up to 14 days or as a sterile graft for up to two years. Methods of using an isolated Dua's layer include on the ocular surface, mid-stroma, on the posterior cornea, and seeded with endothelial cells for corneal application.

### TECHNICAL FIELD AND BACKGROUND

The present technology relates to methods of isolating the Dua's layer and using an isolated Dua's layer in subsequent surgical procedures.

Ophthalmic tissue transplantation uses surgical techniques such as endothelial keratoplasty (“EK”) where a layer of the thin cornea stroma is removed from a donor cornea and transplanted into a recipient patient's eye. Such techniques may use the Descemet's membrane (“DM”) with a layer of endothelial cells or may use the Bowman's membrane as a tissue graft. Existing grafts may be fragile and lack structural integrity, which can make surgical implantation difficult and result in post-surgical complications if the graft is damaged or compromised. Grafts using the Descemet's membrane require decellularization where endothelial cells must be removed before transplant, which increases the risk of mechanical tear and damage and can add significant time and complexity to the procedures.

It would be advantageous to provide surgical techniques that uses the Dua's layer of the cornea as part of a surgical graft. The Dua's layer has superior mechanical characteristics, such as high density, strength, and imperviousness to air, that make the layer well suited for use as a tissue graft. The Dua's layer is also acellular and does not include endothelial cells that must be removed before transplant. A barrier to using the Dua's layer is that it is difficult to isolate and extract from a donor patient, which has limited its usefulness as an ophthalmic tissue graft.

To address the drawbacks of existing surgical techniques and treatment methods, disclosed herein are methods for isolation and removal of the Dua's layer as well as subsequent use of the Dua's layer in surgical ophthalmic tissue graft procedures. The isolated Dua's layer can be preserved as either a fresh graft for up to 14 days or as a sterile graft for up to two years.

### SUMMARY

Disclosed herein are methods for preparing a tissue graft that utilizes an isolated Dua's layer. The method includes the step of using physical peeling, hydrodissection, air dissection, or femtosecond laser dissection to remove a Descemet's membrane from a cornea and/or to dissect the Dua's layer. In one embodiment, the Descemet's membrane is separated from a cornea by inserting a needle into a sclera portion of the cornea, wherein the needle is coupled to a syringe filled with a fluid, and injecting the fluid from the syringe through the needle into a stroma portion of the cornea to create a blister. An amount of fluid is injected to expand the blister until the Descemet's membrane is separated. The Descemet's membrane can then be removed from the cornea using a tool to manually lift the Descemet's membrane from the cornea. The Dua's layer is separated from the cornea using a technique selected from injection of fluid underneath the Dua's layer, or using a femtosecond laser to dissect the Dua's layer.

The needle can be inserted into the sclera portion from the posterior surface of the limbus until the needle reaches the stroma. The fluid can be air or a liquid, such as saline, a corneal storage solution, or a buffered solution. The Dua's layer can be preserved by storing it in a container maintained at a temperature below 32 degrees Fahrenheit below or a container that is at least partially filled with human serum albumin solution at room temperature, or stored as a dry. The Dua's layer can be irradiated to disinfect the layer for storage.

The Dua's layer can be transplanted to an ocular surface of a patient in need. In particular, the Dua's layer can be transplanted as an onlay or inlay to address mild to severe keratoconus. The Dua's layer can also be used as a substrate to promote epithelial and endothelial cell proliferation prior to being transplanted to an ocular surface of a patient.

In one embodiment, the Dua's layer is transplanted to the mid-stroma, and the Dua's layer flattens an anterior corneal surface. In other embodiment, the Dua's layer is transplanted to on anterior cornea, and the Dua's layer flattens an anterior corneal surface. In other techniques, the Dua's layer is transplanted to an posterior cornea with seeded endothelial cells. In yet other techniques, the Dua's layer is transplanted to a anterior cornea, and the Dua's layer is seeded with epithelial cells or periphery epithelial cells have migrated onto the surface of the Dua's layer.

In another embodiment, the method includes a dual blister technique. There, a needle is inserted into a stroma at about 3.0 millimeters to 3.5 millimeters from the limbus of a cornea, wherein the needle is coupled to a syringe filled with fluid. The fluid is injected from the syringe through the needle to create a first blister under the Dua's layer. The fluid leaks under the edges of the Dua's layer and under the Descemet's membrane to create a second blister. An amount of fluid is injected to expand the first blister until the Dua's layer separates from the stroma and to expand the second blister until the Descemet's membrane separates from the Dua's layer.

In yet another embodiment, a surgical tool is used to peel the Descemet's membrane from a cornea. Then a needle is inserted into the sclera where the needle is coupled to a syringe filled with a fluid. The fluid is injected from the syringe through the needle to create a blister under the Dua's layer. An amount of fluid is injected to expand the blister until the Dua's layer separates from the stroma.

In yet another embodiment, a surgical tool is used to peel the Descemet's membrane from a cornea. Then a surgical tool as a donor trephine used to score stroma and another surgical instrument is used manually to peel the Dua's layer

During surgery, an incision can be created in a cornea. The Dua's layer can be injected into the anterior chamber of the cornea using a surgical injector. Fluid is released from the anterior chamber to flatten the anterior chamber, and the surgeon taps the surface of the anterior chamber until the Dua's layer is appropriately positioned and unscrolled. Finally the suture is closed.

### BRIEF DESCRIPTION OF THE DRAWINGS

The drawings described herein are for illustrative purposes only of selected embodiments and not all possible implementations and are not intended to limit the scope of the present disclosure.

FIG. 1A depicts the anatomy of a human eye.

FIG. 1B shows a vertical section of the human cornea.

FIG. 2 shows the separated Dua's layer under electron microscope.

FIG. 3 shows an illustration of the DMEK and PDEK technique in which the Dua's layer, Descemet's membrane and endothelium are replaced with a graft.

FIG. 4 shows an illustration of the Bowman's layer transplantation technique in which the graft is transplanted within the stroma.

FIG. 5A shows a needle horizontally inserted into the sclera and moving toward the limbus.

FIG. 5B shows Trypan Blue injected into the stroma.

FIG. 5C shows Optisol GS injected into the stroma.

FIG. 5D shows the separation of the Descemet's membrane from limbus to limbus using the hydro dissection blister technique.

FIG. 6 shows needle in stroma approximately 3.0-3.5 mm from the limbus and the blister created Dua's layer separation.

FIG. 7 shows optical coherence tomography (“OCT”) image of the blister created by the Dua's layer and Descemet's membrane separation.

### DETAILED DESCRIPTION

The following description of the technology disclosed herein provides examples of the subject matter, methods of performing the techniques described, and methods of using the techniques and inventions disclosed. This description is not intended to limit the scope, application, or uses of any specific invention disclosed in this application, in such other applications that claim priority to this application, or patents issuing therefrom. Regarding the methods disclosed, the ordering of steps for performing the methods are provided as examples and are not intended to be limiting. The order of steps can be different in various embodiments.

“A” and “an” as used herein indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that may arise from ordinary methods of measuring or using such parameters.

The disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific example values for a given parameter may define endpoints for a range of values that may be claimed for the parameter. For example, if Parameter X is described as having the value “A” and a value “Z,” then it is envisioned that Parameter X may have a range of values from about A to about Z. Similarly, the disclosure of two or more ranges of values for a parameter (whether such ranges are nested, overlapping, or distinct) subsume all possible combination of ranges for the value that might be claimed using endpoints of the disclosed ranges. To illustrate, if an example embodiment describes Parameter X as having values in the range of 1 to 10, 2 to 9, or 3 to 8, it is also envisioned that Parameter X may have other ranges of values, such as 1 to 9, 1 to 8, 2 to 6, 4 to 9, among other ranges within the values disclosed.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “anterior,” “posterior,” “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the corneal tissue in use or operation in addition to the orientation depicted in the figures. For example, if the corneal tissue in the figures is turned over, elements described as “below” or “beneath” other elements or features would then be oriented “above” the other elements or features. Thus, the example term “below” can encompass both an orientation of above and below. The corneal tissue may be otherwise oriented (rotated 90 degrees or at other orientations) and the spatially relative descriptors used herein interpreted accordingly.

The present application discloses methods for isolating the Dua's layer using a variety of techniques, such as through the use of a hydrodissection blister technique and gas bubble or through use of a femtosecond or excimer laser, and manual separation. The present application further discloses methods for using the isolated Dua's layer to treat any anterior cornea defects of instability, including, without limitation, keratoconus, stabilization of the anterior cornea after radial keratotomy, management of anterior cornea scars, and epithelial defects caused by a variety of conditions.

The Dua's layer's unique structure, tissue strength, and smooth surface shows promising indications for the treatment of vision disorders. In one application, the Dua's layer graft tissue can be implanted as an onlay or inlay to address mild to severe keratoconus to control continuous thinning and protrusion of the cornea. In an alternative example application, the Dua's layer graft can be used as a substrate to promote epithelial and endothelial cell proliferation and healing.

### Anatomy of the Cornea

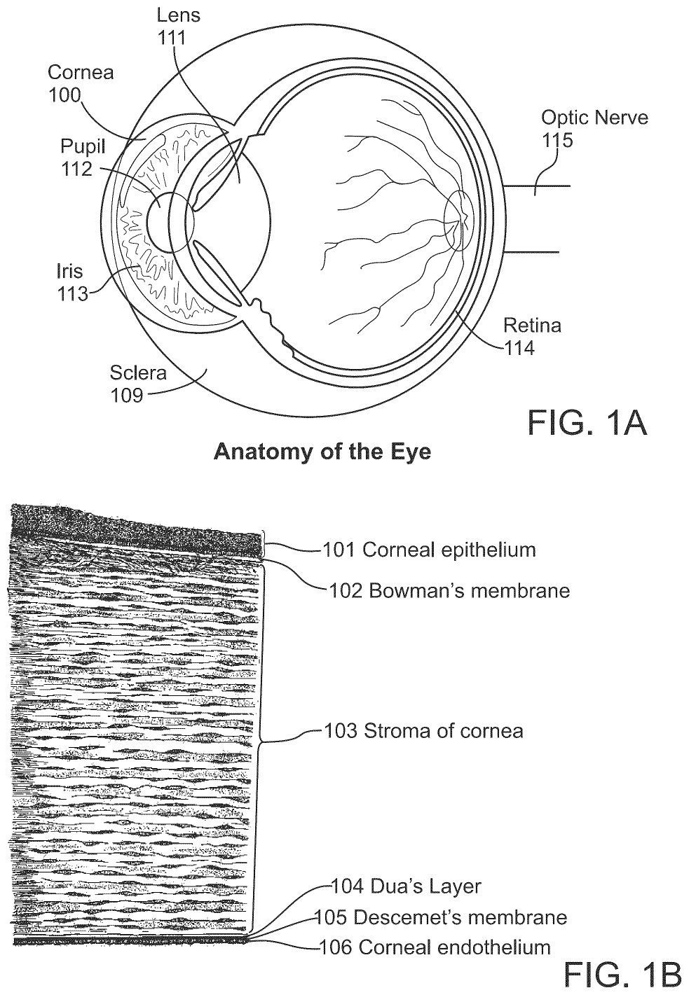

The example human eye shown in FIG. 1A includes a lens 111, cornea 100, pupil 112, iris 113, sclera 109, retina 114, and optic nerve 115. Each part works in concert to provide a person with vision. The lens 111 focuses light entering your eye and directs it to the back of your eye. The cornea 100 protects the inside of the eye and bends light as it enters the eye. The pupil 112 control how much light enters the eye. The iris 113 contains the muscles that control the size of the pupil and is also responsible for eye color. The sclera 109 is the white part of the eye that forms the general shape and structure of the eyeball. The retina 114 is a light-sensitive layer of tissue at the back of the eye that converts light into nerve signals. The optic nerve 115 is a bundle of nerve fibers that transmits visual information from the retina 114 to the brain.

The cornea 100 is a transparent avascular tissue that acts as a structural barrier and protects the eye against infections. An example cornea 100 is show in FIG. 1A. The cornea 100 is made up of cellular and acellular components. The cellular components include the epithelial cells, keratocytes, and endothelial cells. The acellular component includes collagen and glycosaminoglycans. The layers of the cornea are shown in FIG. 1B and include the epithelium 101, Bowman's membrane 102, stroma 103, Dua's Layer 104, Descemet's membrane 105, and endothelium 106.

The corneal epithelium 101 is the outermost layer of the surface of the eye. It usually contains about five to seven layers of cells and is about 50 microns (μm) thick, regenerates quickly, and acts as a barrier to the outside world. The Bowman's layer 102 sits between the epithelium 101 and the stroma 103. The Bowman's layer 102 is eight to fourteen microns thick and is largely involved in maintaining the proper shape of the cornea 100.

The stroma 103 sits between the Bowman's layer 102 and the Descemet's membrane 105 and Dua's layer 104. The stroma 103 is the bulk of the cornea 100 at about 500 microns thick and is filled with collagen fibers that are extremely regular in arrangement and constant in shape. The Descemet's membrane 105 sits between the endothelium 106 and the stroma 103 and Dua's layer 104. The Descemet's membrane 105 is a thin layer at about 5 to 10 microns thick, is secreted by the endothelium 106, and acts as a bed for the endothelial cells to rest on. The Descemet's membrane 105 is composed of various kinds of collagen. The endothelium 106 is a single layer of cells that helps maintain fluid equilibrium inside the stroma 103.

The Dua's layer 104, also called the “pre-Descemet's membrane,” lies anterior to the Descemet's membrane 105 in the cornea 100, sandwiched between the stroma 103 and the Descemet's membrane 105. The Dua's layer 104 has historically been difficult to isolate as the boundaries of the Dua's layer 104 are unclear and not well defined. While the Descemet's membrane 105 extends across the diameter of the cornea 100, the Dua's layer 104 extends only partially across the diameter of the cornea and may have a diameter that is only about three-fourths of the entire cornea diameter. The Dua's layer 104 is thicker toward the center and becomes thinner along the edges of the circumference where the boundaries essentially disappear. Given that the boundaries of the Dua's layer 104 can be difficult to detect, isolating the Dua's layer 104 has proven to be a challenge. Thus, conventional surgical techniques have not utilized the Dua's layer 104 as a tissue graft but instead focused on utilizing the Descemet's membrane 105, and in some cases, the Bowman's membrane 102.

Applicant has isolated the Dua's layer 104 by injecting air or liquid into corneal grafts to carefully separate the layers of the cornea 100. Applicant has also isolated the Dua's layer 104 through femtosecond laser, and manual isolation. The Dua's layer 104 is between 10 microns to 15 microns thick and made predominately of type I and some type VI collagen with abundant elastin (more than any other layer of the cornea 100). The Dua's layer 104 is dense, tough tissue with a high tensile strength and a bursting pressure around 700 mm to 900 mm Hg and can withstand pressures of up to 200 kPa. The Dua's layer 104 is impervious to air and is almost acellular without the presence of endothelial cells. The Dua's layer 104 has also shown the ability to promote cell proliferation.

Overall, the Dua's layer 104 shows favorable strength and pressure resistance as compared to the Descemet's membrane, and use of the Dua's layer 104 does not require decellularization (i.e., removal of the corneal endothelium) prior to use in surgical procedures. Compared to the Descemet's membrane, the Dua's layer 104 is thicker, more stable, and easier to handle during surgical procedures. The Dua's layer 104 can retain its shape while avoiding being structurally compromised during surgical procedures in circumstances where the Descemet's layer 105 might crumble.

Considering the favorable properties of the Dua's layer 104, the Dua's layer 104 is an important discovery with wide-ranging implications. The Dua's layer 104 permits new surgical procedures, such as the pre-Descemet's endothelial keratoplasty. The Dua's layer 104 can be used in anterior and posterior lamellar corneal transplant surgery while making surgery safer with better outcomes. For anterior transplant applications, the Dua's layer 104 is seeded with epithelial cells or epithelial cells in the periphery will migrate to the Dua's layer 104 surface prior to surgical procedures, and for posterior transplant applications, the Dua's layer 104 is seeded with endothelial cells or endothelial cells in the periphery will migrate to the Dua's layer 104 surface prior to surgery.

The Dua's layer 104 can be used to treat a wide variety of anterior cornea defects of instability as well as defects that impact visual acuity for which the Dua's layer has the advantages of tissue strength and a clean, smooth surface. As one example, the Dua's layer 104 is used as an onlay or inlay to treat keratoconus. The occurrence of keratoconus is characterized by a thinning cornea that results in a bulge and an irregular cone-shape in the patient's eye that significantly impacts vision. The strength of the Dua's layer 104 allows the layer to be placed as an onlay over the cornea, similar to the fit of a contact lens, such that the Dua's layer 104 adds rigidity and support to the cornea to prevent bulging and help the cornea maintain its regular shape. The Dua's layer 104 can also be used to treat keratoconus by using the Dua's layer 104 as an inlay where a pocket is formed in the middle of the stroma, and the Dua's layer 104 is inserted in the pocket to help maintain the shape of the patient's eye.

Surgical techniques that position the Dua's layer over the cornea can be used to treat additional conditions, such as repairing corneal scarring caused by diseases like herpes or reducing fluctuations in visual acuity and refractive error that commonly occur following radial keratotomy surgery. During such procedures, the donor recipient's epithelium is removed from the corneal surface, and a Dua's layer graft is positioned onto the recipient cornea. Due to inherent tissue properties, after removed from the donor cornea a graft may naturally scrolls tightly backwards on itself. Surgeons must insert the graft into anterior chamber using a needle injector and then unscroll the graft (make it flat) as part of the transplant procedure. The options for unscrolling the ophthalmic tissue include repeatedly tapping on the outer cornea, injecting air or fluid inside the eye, or manipulating the tissue further with surgical instrumentation.

Those of skill in the art will appreciate the above examples are non-limiting, and use of the Dua's layer as a corneal graft finds utility in treating a variety of epithelial defects caused by mechanical trauma, corneal dryness, neurotrophic disease, infection, or other conditions.

### Conventional Surgical Techniques

Previous transplant procedures requiring replacing the entire thickness of the cornea 100. Today, surgeons can now replace specific layers of the cornea 100, such as the front layers and back layers of the cornea 100, in addition to the entire cornea 100 itself. Transplanting less tissue helps preserve the integrity of the eye, reduce the risk of failure or rejection of the graft, and speeds up the recovery period.

Endothelial keratoplasty is a cornea transplant technique that is the preferred way to restore vision when the inner cell layer of the cornea 100 stops working properly. Endothelial keratoplasty selectively replaces only the diseased layer of the cornea 100 and leaves the healthy areas intact. A surgeon will remove the diseased inner cell layer of the cornea 100 and implant healthy donor tissue through a small incision. The surgeon positions the donor tissue against the patient's cornea 100 and closes the incision.

Within the last two decades, surgeons have started performing partial thickness keratoplasty, such as the Descemet's Stripping Automated Endothelial Keratoplasty (“DSAEK”), Descemet's Membrane Endothelial Keratoplasty (“DMEK”), Pre-Descemet's Endothelial Keratoplasty (“PDEK”), and Deep Anterior Lamellar Keratoplasty (“DALK”) or Anterior Lamellar Keratoplasty (“ALK”). With changing techniques, there has been a shift from surgeons preparing the tissue grafts to eye banks preparing the tissue grafts. Allowing eye banks to perform the ophthalmic tissue preparation allows surgeons to save time in the operating room and decrease the chances of graft failure.

DMEK is the thinnest EK and utilizes a graft about 10 microns to 15 microns in thickness, and includes only the Descemet's membrane 105 and endothelium 106. PDEK is a thicker EK than DMEK, utilizes graft about 25 microns to 30 microns, and includes the Dua's layer 104, Descemet's membrane 105, and the endothelium 106. The most common technique for preparing DMEK grafts is known as submerged cornea 100 using background away (“SCUBA”). Using the SCUBA technique, a technician carefully scores a circle around the periphery of the cornea 100 before peeling the Descemet's membrane 105 across the cornea. The Descemet's membrane 105 can be separated from the stroma 103 by punching the cornea 100 with a trephine. Disadvantages of the SCUBA technique include touching of the instrument to the cornea, multiple lifting or peeling of the cornea, uneven tension during peeling, created endothelial stress strain, horseshoe-shaped tears, and excessive separation resistance.

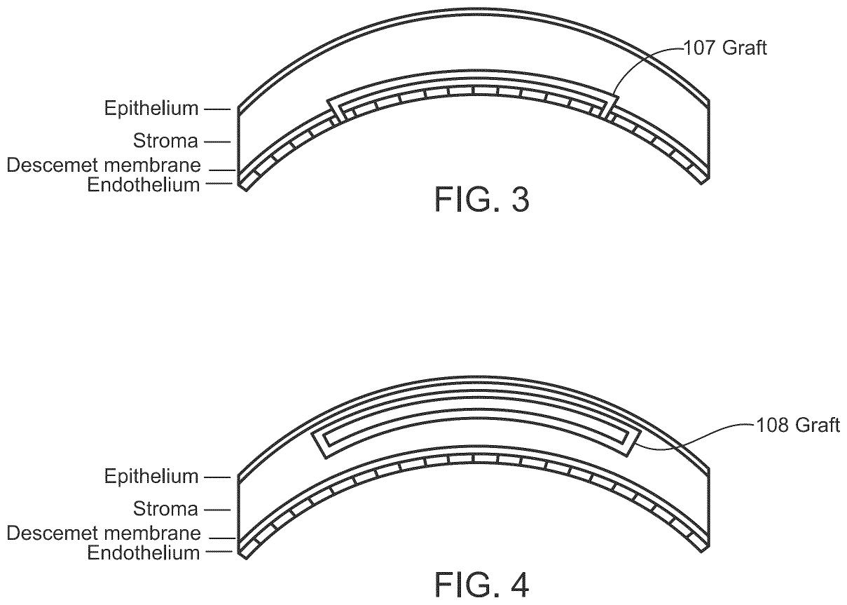

A PDEK graft 107 is primarily prepared by inserting a needle into the cornea 100 and injecting air to create the graft. Once the bubble is made, the cornea 100 is punched with a trephine. The bubble technique can also be used to prepare a DMEK graft. The use of the bubble technique employs a safer no-touch method for graft preparation. The bubble technique applies an even pressure to the tissue and avoids the uneven tension and resulting horseshoe-shaped tears that occur with manual lifting techniques. Grafts from DMEK and PDEK 107 are transferred to the posterior stroma 103 and function to replace the damaged cells of endothelial layer, as shown in FIG. 3.

Extraction and use of the Bowman's membrane 102 has been used to treat keratoconus. Donor tissue preparation for the Bowman's membrane 102 consists of manually peeling the Bowman membrane 102 from the anterior stroma 103 and then manually removing the epithelium 101. Then, a forceps with round edges are used to lift and grasp the peripheral Bowman's membrane 102 edge and peel the Bowman membrane 102 away from the underlying anterior stroma 103. Bowman's membrane grafts 108 are transferred to the mid-stroma 103 and function to pull, strengthen, and make the anterior corneal surface flatter, as depicted in FIG. 4.

### Applicant's Improvement Over Conventional Methods

While the PDEK graft technique has been able to isolate the Dua's layer 104, Descemet's membrane 105, and the endothelium 106, there are no conventional techniques for isolating solely the Dua's layer 104. Due to its relatively recent discovery, the Dua's layer 104 is not well understood, including techniques used to isolate the layer. The boundaries of the Dua's layer 104 are limited, unclear, and not well defined. Notwithstanding, use of the Dua's layer 104 in the treatment of keratoconus and cell generation shows improvement over traditional treatment methods.

Applicant has developed techniques to separate the Dua's layer 104 from the cornea using “bubbles” (injection of air), blistering (injection of liquid), dissection using femtosecond laser, and manual extraction. The techniques utilizing the injection of air or liquid are called “hydrodissection.” Hydrodissection entails the use of fluid (air or liquid) to separate and isolate the Dua's layer 104 from the cornea 100. Hydrodissection is a “no touch” method for Dua's layer graft preparation, which addresses the drawbacks of manual extraction that include tearing and stress to the graft that leads to endothelial loss. Applicant is the first to utilize hydrodissection techniques for isolation of the Dua's layer 104. The hydrodissection techniques described herein are generally described with reference to the injection of liquid to create blisters, but those of skill in the art will appreciate that the techniques are applicable to the use of air to create bubbles during ophthalmic tissue isolation and extraction.

In one embodiment, the Descemet's membrane 105 is separated from the donor cornea 100 by first removing the Descemet's membrane 105 from the cornea 100 leaving the Dua's layer 104 on the cornea 100. Then, the Dua's layer 104 is separated from the cornea 100. The Descemet's membrane 105 is separated from the cornea 100 by manual peeling (e.g., the SCUBA technique) or by injecting liquid, air, or both into the cornea 100. For hydrodissection, the fluid can be saline, corneal storage solution such as Optisol-GS, Eusol-C, Kerasave, or Cornea Cold, or any buffered solution. Buffered corneal solutions contain various components, including buffers, electrolytes, nutrients, and antibiotics, to support endothelial cells and ensure the cornea remains viable for a certain period. Commonly used buffer solutions for corneal storage include HEPES buffer and phosphate-buffered saline. Once the Descemet's membrane 105 is removed, the Dua's layer 104 is separated from the cornea 100 by either injecting air or fluid into the cornea 100 or by manually peeling the Dua's layer 104 away from the cornea 100.

In other embodiments, a femtosecond laser is used to perform one or both steps of removing the Descemet's membrane from the cornea and separating the Dua's layer 104 from the cornea 100. Femtosecond lasers operate in the near-infrared region and emit ultrashort pulses. Power is a function of time, so using pulses rather than a continuous beam reduces the power needed to make a cut, thereby causing less thermal damage to the surrounding tissue. Femtosecond lasers produce a cut rather than a full ablation such as an excimer laser that destroys tissue. Various parameters control the femtosecond laser cut, including cutting angle, trephination diameter (diameter of the cut), anterior uncut depth, uncut area thickness, cut speed, repetition rate, pulse duration, and energy percentage.

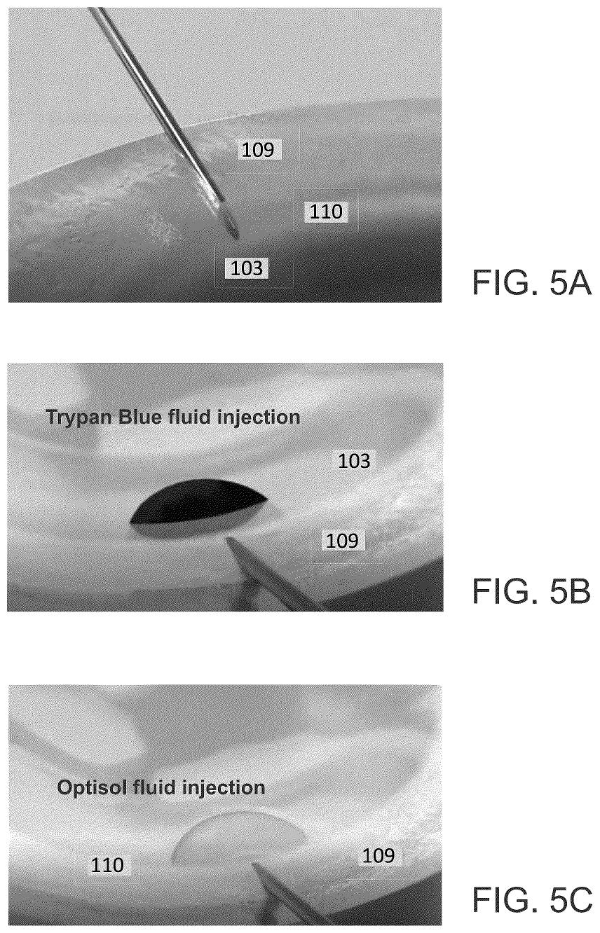

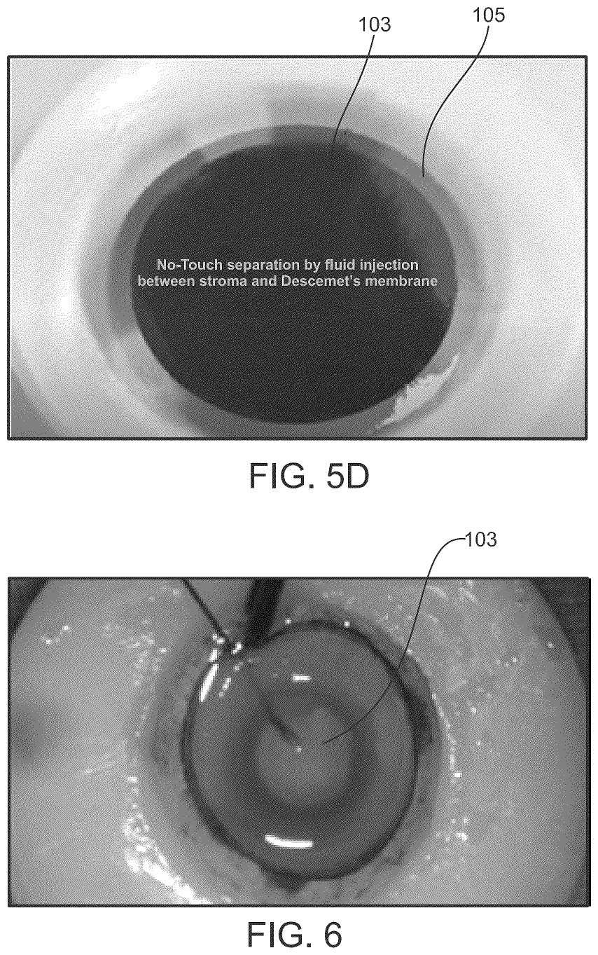

In another embodiment, the Descemet's membrane 105 is first removed from the cornea 100 by the hydrodissection blister technique. A donor cornea with the endothelial surface up is placed on a Teflon punch block, and the endothelium 106 is first stained with Trypan Blue as seen in FIGS. 5A and 5B. Simultaneously, fluid is injected into the inferior part of stroma 103 by horizontally inserting an approximately 27 to 30 gauge needle attached to a 1 cc syringe with Optisol GS or Balance Salt Solution. The needle is inserted into the sclera 109 approximately 2 mm outside of the limbus 110 and moved forward as seen in FIG. 5C. The fluid is injected into the stroma 103 with soft pressure where the needle is passed 1.5 mm to 2 mm from the limbus 110 into the stroma 103 to create a blister. The blister continues to expand and separates the Descemet's membrane 105 until the blister reaches the limbus 110, as seen in FIG. 5D. Then, air, fluid, or manual peeling is used to separate the Dua's layer 104 from the cornea 100. Alternatively, the Dua's layer 104 can be dissected using a femtosecond laser.

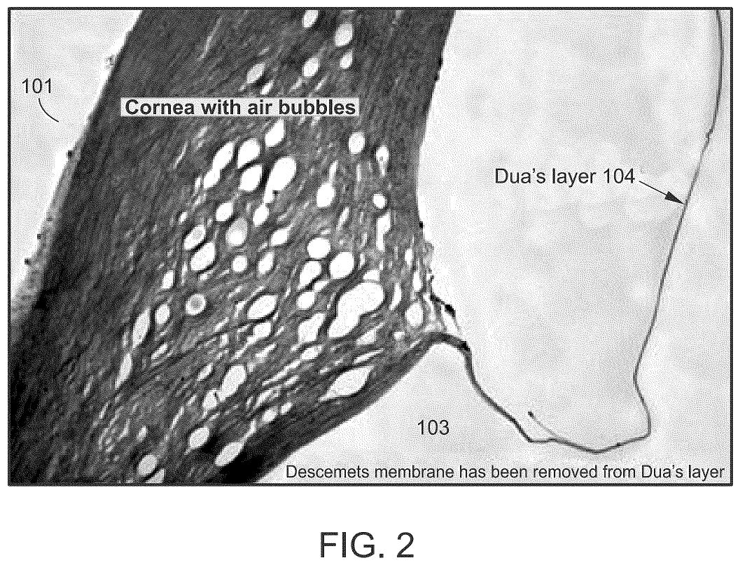

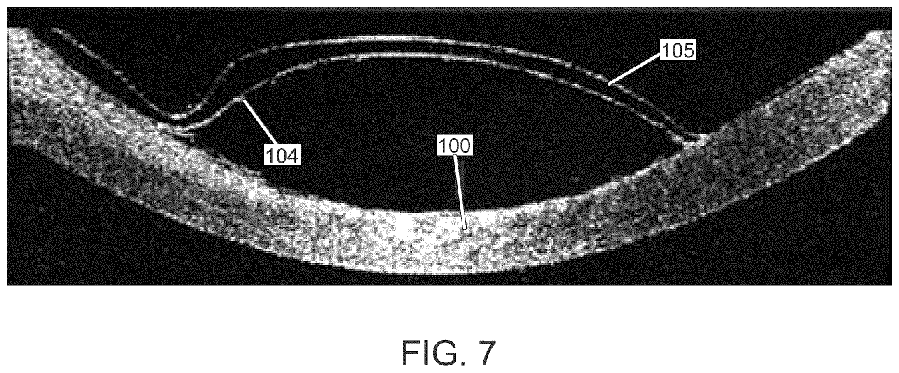

In another embodiment, a needle is inserted into the inferior stroma 103 at about 3.0 mm to 3.5 mm from the limbus (see FIG. 6), and fluid is injected under the Dua's layer 104. The injection depth is close to the interior side of the stroma such that the final depth can be approximately 90% of the way through the thickness of the stroma nearly reaching the anterior chamber. As fluid is injected, fluid fills the interior side of the Dua's layer 104 but leaks from under the edges of the Dua's layer 104 because of its thin edges. The fluid that leaks from the edges of the Dua's layer 104 then fills the interior of the Descemet's membrane 105. Fluid simultaneously fills underneath the Dua's layer 104 and the Descemet's membrane 105 (see FIG. 7), which creates two blisters at the same time with one on top of the other. One blister causes the Dua's layer 104 to separate from the stroma 103 (for visualization refer to FIGS. 2 and 7), and the second blister causes the Descemet's membrane 105 to separate from the Dua's layer 104. The Dua's layer 104 can then be removed from the cornea 100.

To test the liquid blister separation method, Applicant prepared 104 research corneas and 44 transplant donor corneas using the blister separation method. Descemet's membrane separation was achieved in all cases. The diameter of the separated Descemet's membranes was 9.5 millimeters on average. The mean donor endothelial cell density prior to preparation was 2824±131 cells per square millimeter, and the cell density post preparation was 2893±121 cells per square millimeter. The cell density measurements confirmed that the membranes were successfully removed without damaging the issue, and staining techniques were used to confirm viable endothelium.

Once the Dua's layer 104 is isolated, it can be provided to surgeons as either: (i) a fresh preserved graft that can be stored in cornea preservative on ice for up to 14 days; or (ii) as a sterile graft that can be stored in human serum albumin, irradiated for sterilization, and stored at room temperature for up to two years. Irradiation can use gamma or e-beam radiation for sterilization purposes. Gamma-irradiated tissues are subject to greater exposure times than e-beam-treated tissue because gamma irradiation dose rates are much lower than those used for e-beam irradiation, thus requiring longer exposure times. E-beam irradiation exposes the tissues to high dose rates of irradiation at a low penetration for much shorter exposure times, making the e-beam process more effective and efficient. A minor disadvantage posed by the longer exposure periods of gamma-irradiated tissues, however, is the formation of minor changes in corneal collagen matrix. E-beam irradiation has not been found to result in noticeable differences in the collagen matrix of corneas, thereby leaving important clinical properties such as clarity and light scattering unaffected.

Current grafting techniques, including DMEK, PDEK, and Bowman's membrane grafts, are not the best substrate for the treatment of keratoconus. The Dua's layer 104, however, is a more advantageous alternative as it has shown to be ideal for anterior corneal clinical indications that require strength and cell overgrowth. The Dua's layer 104 is thicker and easier to isolate than the Bowman's layer 102 with a smoother surface, and it has a high tensile strength that allows the layer to resist tearing. The Dua's layer 104 is impervious to air, and has more elastin than any other layer in the cornea 100. Such unique qualities make the Dua's layer 104 a better choice for use in anterior cornea 100 application. The Dua's layer 104 can be used as a scaffold to promote epithelial cell proliferation and healing within the cornea 100.

Additionally, the Dua's layer 104 has the potential to be transplanted onto the ocular surface as an onlay. The Dua's 104 layer may be grafted onto the cornea and used to treat anterior cornea surface issues including, without limitation epithelial defects caused by mechanical trauma, corneal dryness, neurotrophic disease, infection, scarring, post-post-surgical changes, or other conditions. The Dua's layer may also cornea instability and thinning (e.g., keratoconus) by using the Dua's layer as scaffold, carrier and/or reinforcement that support anterior cornea health and strengthens the unstable or thinning cornea 100.

Furthermore, the Dua's layer 104 may be inlayed mid-stroma 103 and can function to pull, strengthen, and make the anterior corneal surface flatter. One advantage of the Dua's layer 104 over the Bowman's membrane 102 is that the Dua's layer 104 is thicker and, therefore, provides stronger structural support. A further benefit of using a Dua's layer graft is that the Dua's layer does not need to be decellularized, unlike a Bowman's membrane graft or the Descemet's membrane 105.

Applicant's technique produces a decellularized Dua's layer 104 without additional steps, time, or materials. Most protocols suggested for decellularization are very time consuming and can take multiple days. Traditional decellularization methods use mechanical, enzymatic and or chemical for decellularization. Decellularized tissue possess tissue-specific three-dimensionality and can be used as a cell-free scaffold of an intact extracellular matrix for subsequent cellular repopulation. The Dua's layer 104, much like the Descemet's membrane 105, can be used as a cell culture substrate. The Dua's layer 104 can be used to support the proliferation of limbal stem cells (also known as corneal epithelial stem cells). This application can be used to regenerate the damaged corneal surface in patients with limbal stem cell deficiency. Limbal stem cell deficiency can cause ocular pain from corneal erosions and decreased vision from stromal scarring or epithelial irregularity.

Example embodiments are provided so that this disclosure will be thorough, and will fully convey the scope to those who are skilled in the art. Numerous specific details are set forth such as examples of specific methods and uses to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions and methods can be made within the scope of the present technology, with substantially similar results.

What is claimed is:

1. A method for processing a tissue graft comprising:
(a) separating a Descemet's membrane from a cornea by
(i) inserting a needle into a sclera portion of the cornea, wherein the needle is coupled to a syringe filled with a fluid, and
(ii) injecting the fluid from the syringe through the needle into a stroma portion of the cornea to create a blister, wherein an amount of the fluid is injected to expand the blister until the Descemet's membrane is separated;
(b) removing the Descemet's membrane from the cornea using a tool to manually lift the Descemet's membrane from the cornea; and
(c) separating a Dua's layer from the cornea using a technique selected from (i) injection of fluid underneath the Dua's layer, (ii) using a femtosecond laser to dissect the Dua's layer, or (iii) manual separation.

2. The method for processing a tissue graft of claim 1, wherein the needle is inserted into the sclera portion from the posterior surface of the limbus until the needle reaches the stroma.

3. The method for processing a tissue graft of claim 1, wherein the fluid is air.

4. The method for processing a tissue graft of claim 1, wherein the fluid is a liquid selected from saline, a corneal storage solution, or a buffered solution.

5. The method for processing tissue grafts of claim 1, wherein the Dua's layer is preserved by either:
(a) storing the Dua's Layer in a first container that is maintained at a temperature below 32 degrees Fahrenheit below; or
(b) storing the Dua's layer in a second contained at least partially filled with human serum albumin solution and irradiating the Dua's layer.

6. The method for processing a tissue graft of claim 5, wherein the Dua's layer is removed from the first or second container and transplanted to an ocular surface of a patient in need thereof.

7. The method for processing a tissue graft of claim 1, wherein the Dua's layer is implanted as an onlay or inlay to address mild to severe keratoconus.

8. The method for processing a tissue graft of claim 1, wherein (a) the Dua's layer is used as a substrate to promote epithelial and endothelial cell proliferation;
(b) and the Dua's layer is transplanted to an ocular surface of a patient.

9. A method for a processing a tissue graft comprising:
(a) inserting a needle into a stroma at about 3.0 millimeters to 3.5 millimeters from the limbus of a cornea, wherein the needle is coupled to a syringe filled with a fluid, and
(b) injecting the fluid from the syringe through the needle, wherein
(i) injection of the fluid creates a first blister under the Dua's layer, and
(ii) the fluid leaks under the edges of the Dua's layer and under a Descemet's membrane to create a second blister; and
(iii) an amount of the fluid is injected to (A) expand the first blister until the Dua's layer separates from the stroma, and (B) expand the second blister until the Descemet's membrane separates from the Dua's layer.

10. The method for processing a tissue graft of claim 9, wherein the Dua's layer is preserved by either:
(a) storing the Dua's Layer in a first container that is maintained at a temperature below 32 degrees Fahrenheit below; or
(b) storing the Dua's layer in a second container at least partially filled with human serum albumin solution and irradiating the Dua's layer.

11. The method for processing a tissue graft of claim 10, wherein the Dua's layer is removed from the first or second container and transplanted to an ocular surface of a patient in need thereof.

12. The method for processing tissue grafts of claim 10, wherein:
(a) the Dua's layer is removed from the first or second container;
(b) the Dua's layer is and transplanted to the mid-stroma; and
(c) the Dua's layer flattens an anterior corneal surface.

13. The method for processing a tissue graft of claim 10, wherein the Dua's layer is removed from the first or second container and transplanted to an anterior cornea.

14. The method for processing a tissue graft of claim 13, wherein the Dua's layer is seeded with endothelial cells or periphery endothelial cells have migrated onto the Dua's layer.

15. The method for processing a tissue graft of claim 10, wherein the Dua's layer is removed from the first or second container and transplanted to a posterior cornea.

16. The method for processing a tissue graft of claim 15, wherein the Dua's layer has been seeded with epithelial cells or periphery epithelial cells have migrated onto the Dua's layer.

17. A method for a processing a tissue graft comprising:
(a) using a surgical tool to peel a Descemet's membrane from a cornea;
(b) inserting a needle into a sclera, wherein the needle is coupled to a syringe filled with a fluid, and
(c) injecting the fluid from the syringe through the needle, wherein
(i) injection of the fluid creates a blister under the Dua's layer, and
(ii) an amount of the fluid is injected to expand the blister until the Dua's layer separates from the stroma.

18. The method for processing a tissue graft of claim 17 further comprising the steps of:
(a) creating an incision in a cornea;
(b) injecting the Dua's layer into the anterior chamber of the cornea;
(c) releasing fluid from the anterior chamber to flatten the anterior chamber;
(d) tapping and swiping on the surface of the anterior chamber until the Dua's layer is appropriately positioned and unscrolled; and
(e) closing the incision with a suture.

19. The method for processing a tissue graft of claim 18, wherein the Dua's layer is implanted as an onlay or inlay to treat mild to severe keratoconus.

20. The method for processing a tissue graft of claim 18, wherein the Dua's layer is used as a substrate to promote epithelial and endothelial cell proliferation.
